(19) United States
(12) Patent Application Publication
(10) Pub. No.: US 2009/0130022 A1
(43) Pub. Date: May 21, 2009

(54) MRI CONTRAST MEDIUM CONTAINING LIPOSOMES

(75) Inventors: Junji Nishigaki, Kanagawa (JP); Hiroyuki Hirai, Kanagawa (JP); Masayoshi Kojima, Kanagawa (JP); Kazuhiro Aikawa, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/916,391

(22) PCT Filed: Jun. 6, 2006
(86) PCT No.: PCT/JP2006/311701

§ 371 Date: Dec. 3, 2007

(30) Foreign Application Priority Data
Jun. 6, 2005 (JP) 2005-165722

(51) Int. Cl. A61B 5/055; A61K 9/127

(52) U.S. Cl. 424/9.3; 424/450

(57) ABSTRACT

A liposome containing a phosphatidylcholine and a phosphatidylserine in combination as membrane components at a phosphatidylcholine:phosphatidylserine molar ratio of 3:1 to 1:2, and said liposome containing superparamagnetic particles having a mean particle size not less than 1 nm and not more than 50 nm. A means for selectively accumulating an MRI contrast medium in a lesion of a vascular disease caused by abnormal proliferation of vascular smooth muscle cells such as arteriosclerosis and restenosis after PTCA is provided.

### TECHNICAL FIELD

The present invention relates to a liposome containing a combination of a phosphatidylcholine and a phosphatidylserine as membrane components, and more specifically, the present invention relates to a liposome containing a combination of a phosphatidylcholine and a phosphatidylserine as membrane components and superparamagnetic particle.

The present invention also relates to an MRI contrast medium comprising the liposomes containing a combination of a phosphatidylcholine and a phosphatidylserine as membrane components and superparamagnetic particles.

### BACKGROUND ART

In recent years, the NMR imaging method (MRI), which captures various pathological lesions as images, has been focused as one of non-invasive and non-destructive clinical diagnostic methods. Ordinary MRI measurements often requires use of an MRI contrast medium to enhance contrast between a pathological lesion and a normal tissue. Therefore, many researches on MRI contrast media have been made so far.

Major imaging parameters of MRI that can be controlled with a contrast medium include spin-lattice relaxation time (T1) and spin-spin relaxation time (T2). For example, when a paramagnetic chelate comprising manganese (2+), gadolinium (3+), or iron (3+) as a base agent is used as an MRI contrast medium, the spin-lattice relaxation time (T1) is decreased, and thereby the signal intensity can be increased. An MRI contrast medium using magnetic/superparamagnetic particles as a base agents decreases the spin-spin relaxation time (T2) and thus causes decrease of the signal intensity. Massive administration of a paramagnetic chelate or a paramagnetic compound comprising dysprosium as a base material also decreases the MR signal intensity. The details of these MRI contrast media are described as a review in, for example, Non-patent document 1.

There are also many recent reports of applications of hydrophilic chelate compounds as MRI contrast media, such as GdDTP [gadolinium(III)-diethylenetriamine-N,N,N′,N″,N″-pentaacetate complex], GdDOTA [gadolinium(III)-1,4,7,10-tetraazacyclododecane-1,4,7,10-tetraacetate complex], GdHPDO3A [gadolinium(III)-10-(2-hydroxypropyl)-1,4,7,10-tetraazacyclododecane-1,4,7-triacetate complex] and GdDTPA-BMA {gadolinium(III)-[N,N-bis[2-[(carboxymethyl)(methylcarbamoyl) methyl]amino]ethyl]glycinate complex}. These hydrophilic chelate compounds are extracellularly distributed and excreted from the kidney. Such compounds are useful for, for example, visualizing pathological lesions of the central nerve system. Examples of contrast media particularly specific to organs or tissues include MnDPDP (manganese(II)-N,N-dipyridoxylethylenediamine-N,N-diacetate-5,5-bis(phosphate) complex), paramagnetic porphyrin, and the like.

Furthermore, uses of liposomes encapsulating various paramagnetic metal ions and chelates as MRI contrast media have been reported. For example, small monolayer liposomes (small unilamellar vesicles, SUV), large monolayer liposomes (large unilamellar vesicles, LUV), and multilayer liposomes (multilamellar vesicles, MLV) having variety of lipid compositions, surface charging degrees and particle sizes have been proposed as MRI contrast media that specifically accumulate in pathological sites (see, Patent document 1 and Non-patent documents 2 to 10). However, liposome MRI contrast media are easily trapped by the reticuloendothelial system, and accordingly, they have high accumulation property in the liver. However, they have poor accumulation property in the other pathological lesions (those of vascular diseases, tumors and the like), and also have short blood retention time. Therefore, despite the abundant reports, no product has been launched in the market or developed in the later clinical trial stage.

In the modern society, especially in the societies of advanced countries, opportunities of ingesting high calorie and high fat diet are increasing. For this reason, mortalities due to ischemic diseases resulting from arteriosclerosis (heart diseases such as myocardial infarction and angina pectoris, cerebrovascular diseases such as cerebral infarction and cerebral hemorrhage) have been increasing. Therefore, application of MRI to the diagnoses of vascular diseases has been expected, and an MRI contrast medium that specifically accumulates in vascular lesions has been desired.

As a report of an MRI contrast medium that specifically accumulates in vascular lesions, manganese(III)-α, β, γ, 67 -tetrakis(4-sulfophenyl)porphine chelate (hereinafter abbreviated as “Mn-TSPP”) has been proposed, and the chelate is considered to be suitable for obtaining an MRI image of arteriosclerosis, in particular, a lesion of early stage thereof. However, Mn-TSPP has a low accumulation ratio in a cholesterol lesion which is the major cause of arteriosclerosis, and therefore they fail to provide practically satisfactory images.

Separately from the above reports concerning MRI contrast media, attempts focusing on membrane components of liposomes have also been reported, in which a hydrophobic iodine contrast medium is formulated as a liposome preparation comprising a phospholipid to achieve selective accumulation as an X-ray contrast medium in objective pathological lesions (see, Patent document 2 and Non-patent documents 11 to 13). However, no liposome suitable as an MRI contrast medium is specifically disclosed in these reports.

[Patent document 1] Japanese Patent Unexamined Publication (KOKAI) No. 7-316079
[Patent document 2] Japanese Patent Unexamined Publication (KOKAI) No. 2003-55196
[Non-patent document 1] Magnetic Resonance Imaging, Mosby, Chapter 14, 1992
[Non-patent document 2] Radiology, 171, p. 19, 1989
[Non-patent document 3] Invest. Radiol., 23, p. 131, 1988
[Non-patent document 4] Radiology, 171, p. 77, 1989
[Non-patent document 5] Biochim. Biophys. Acta, 10222, p. 181, 1990
[Non-patent document 6] Invest. Radiol., 25, p. 638, 1990
[Non-patent document 7] Magn. Reson. Imaging, 7, p. 417, 1989
[Non-patent document 8] J. Pharmacol. Exp. Ther., 250, p. 1113, 1989
[Non-patent document 9] Invest. Radiol., 23, p. 928, 1988
[Non-patent document 10] Radiology, 171, p. 81, 1989
[Non-patent document 11] Pharm. Res., 16 (3), p. 420, 1999
[Non-patent document 12] J. Pharm. Sci., 72 (8), p. 898, 1983
[Non-patent document 13] Invest. Radiol., 18 (3), p. 275, 1983

### DISCLOSURE OF THE INVENTION

An object of the present invention is to provide a means for selectively accumulating an MRI contrast medium in a lesion of a vascular disease caused by abnormal proliferation of vascular smooth muscle cells such as arteriosclerosis and restenosis after PTCA. Another object of the present invention is to image a biological environment of a vascular disease or the like by MRI using the aforementioned means.

The inventors of the present invention conducted various studies to achieve the foregoing objects, and as a result, they found that liposomes containing a phosphatidylcholine (hereinafter abbreviated as “PC”) and a phosphatidylserine (hereinafter abbreviated as “PS”) as membrane components selectively accumulated in pathological lesions, and that an MRI contrast medium selectively accumulated in pathological lesions was successfully provided by encapsulating superparamagnetic particles, a T2 enhancing type contrast medium shortening the transverse relaxation time (T2) of proton, in the liposomes as mentioned above. The present invention was achieved on the basis of the aforementioned findings.

The present invention thus provides a liposome containing a phosphatidylcholine and a phosphatidylserine in combination as membrane components at a phosphatidylcholine:phosphatidylserine molar ratio of 3:1 to 1:2. According to a preferred embodiment of the present invention, there is provided the aforementioned liposome, wherein the molar ratio of phosphatidylcholine and phosphatidylserine is 1:1.

According to other preferred embodiments of the present invention, there are provided the aforementioned liposome, which contains superparamagnetic particles having a mean particle size not less than 1 nm and not more than 50 nm; the aforementioned liposome, wherein the superparamagnetic particles are selected from the group consisting of those of iron oxide and ferrite (Fe, M)₃O₄; and the aforementioned liposome, wherein the superparamagnetic particles are those of magnetite, maghemite, or a mixture thereof.

From another aspect of the present invention, there is provided an MRI contrast medium, which comprises the aforementioned liposome according to any one of the aforementioned embodiments. According to preferred embodiments of the invention, provided are the aforementioned MRI contrast medium, which is used for imaging of a vascular disease; the aforementioned MRI contrast medium, which is used for imaging of vascular smooth muscle cells which abnormally proliferate under an influence of foam macrophages; the aforementioned MRI contrast medium, which is used for imaging of a tissue or lesion where macrophages localize; the aforementioned MRI contrast medium, wherein the tissue where macrophages localize is selected from the group consisting of tissues of liver, spleen, air vesicle, lymph node, lymph vessel, and renal epithelium; and the aforementioned MRI contrast medium, wherein the pathological site where macrophages localize is selected from the group consisting of lesions of tumor, inflammation, and infection.

From a further aspect of the present invention, there is provided a method for imaging a vascular disease, which comprises the step of administering the liposome of the present invention according to any one of the aforementioned embodiments to a mammal including human to perform MRI.

The liposomes of the present invention selectively accumulate at a site of a vascular disease caused by abnormal proliferation of vascular smooth muscle cells such as arteriosclerosis and restenosis after PTCA. Therefore, a biological environment of a vascular disease or the like can be imaged by MRI using the liposome of the present invention.

### BEST MODE FOR CARRYING OUT THE INVENTION

The liposome of the present invention contains a combination of a phosphatidylcholine and a phosphatidylserine as membrane components. The content of the combination based on the total mass of the membrane components may be, for example, from 5 to 100 mass %, and preferably be 20 to 100 mass %, more preferably 80 to 100 mass %.

The phosphatidylcholine is not particularly limited. Preferred examples of the phosphatidylcholine include egg PC, dimyristoyl-PC (DMPC), dipalmitoyl-PC (DPPC), distearoyl-PC (DSPC), dioleyl-PC (DOPC) and the like. The phosphatidylserine is not particularly limited. Preferred examples of the phosphatidylserine include those having lipid moieties similar to those of the phospholipids mentioned as preferred examples of the phosphatidylcholines. The molar ratio of PC and PS (PC:PS) used is from 3:1 to 1:2, most preferably 1:1.

As the liposome of the present invention, a liposome containing, besides the phosphatidylcholine and phosphatidylserine, a phosphoric acid dialkyl ester as a membrane component is also preferred. The two alkyl groups constituting the dialkyl ester of phosphoric acid are preferably the same groups. It is sufficient that each group contains 6 or more carbon atoms, and the group preferably contains 10 or more carbon atoms, more preferably 12 or more carbon atoms. The carbon number of the alkyl group may be 24 or less, but is not particularly limited. Preferred examples of the phosphoric acid dialkyl ester include, but not limited to, dilauryl phosphate, dimyristyl phosphate, dicetyl phosphate and the like. In this embodiment, preferred amount of the phosphoric acid dialkyl ester is from 1 to 50 mass %, more preferably from 1 to 30 mass %, further preferably from 1 to 20 mass %, based on the total mass of phosphatidylcholine and phosphatidylserine.

The membrane components of the liposome of the present invention are not limited to the aforementioned components, and other components may be added. Examples of such components include cholesterol, cholesterol esters, sphingomyelin, monosial ganglioside GM1 derivatives [FEBS Lett., 223, 42 (1987); and Proc. Natl. Acad. Sci., USA, 85, 6949 (1988) can be referred to], glucuronic acid derivatives [Chem. Lett., 2145 (1989); and Biochim. Biophys. Acta, 1148, 77 (1992) can be referred to], and polyethylene glycol derivatives described in Non-patent documents 18 and 19 [Biochim. Biophys. Acta, 1029, 91 (1990); and FEBS Lett., 268, 235 (1990) can be referred to]. However, the components are not limited to these examples.

As the method for preparing the liposome of the present invention, any methods known in the field of the art may be used. Examples of the preparation method include the methods described in Ann. Rev. Biophys. Bioeng., 9, 467 (1980), or “Liposomes” (Ed. by M. J. Ostro, MARCELL DEKKER, INC.). Specific examples include, but not limited to, the ultrasonication method, ethanol injection method, French press method, ether injection method, cholic acid method, calcium fusion method, freeze and thawing method, reverse phase evaporation method and the like. Size of the liposome may be any of those obtainable by the aforementioned methods. Generally, a size in average may be 400 nm or less, preferably 200 nm or less. Structure of the liposome is not particularly limited, and may be unilamellar or multilamellar structure.

The liposome of the present invention may be formulated with one or more kinds of components known as MRI contrast media in the inside of the liposome or as membrane components. Preferred examples of the components known as MRI contrast media include superparamagnetic particles. The liposome of the present invention preferably contains superparamagnetic particles having a mean particle size not less than 1 nm and not more than 50 nm. In the liposome of the present invention, the superparamagnetic particles are preferably encapsulated in the hydrophilic portion in the inside of the liposome. Superparamagnetic particles having a mean particle size not less than 1 nm can be stably prepared. For example, where a target substance is an intracellular substance, superparamagnetic particles having a mean particle size not more than 50 nm can penetrate into the inside of cells and capture the target substance. The mean particle size of the superparamagnetic particles is preferably not less than 3 nm and not more than 50 nm, more preferably not less than 5 nm and not more than 40 nm, from viewpoints of stability of crystals and magnetic response.

The superparamagnetic particles mean particles having a property that they are strongly magnetized in the same direction as that of an external magnetic field applied, whilst the magnetization is eliminated when the external magnetic field is removed. Examples of the superparamagnetic particles include those of metal oxides such as iron oxide and ferrite (Fe, M)₃O₄, and those of iron oxide are especially preferred. The term “iron oxide” used herein encompasses magnetite, maghemite, and a mixture of magnetite and maghemite. The superparamagnetic particles may have a core/shell structure where the surface and the inside consists of different materials. M in the aforementioned formula is a metal ion that can be used together with iron ion to form a magnetic metal oxide, and is typically chosen from transition metal ions. Preferred examples of M in the aforementioned formula include Zn²⁺, Co²⁺, Mn²⁺, Cu²⁺, Ni²⁺, Mg²⁺, and the like. A molar ratio of M/Fe is determined according to the stoichiometric composition of the chosen ferrite. The superparamagnetic particles may consist of a salt of the aforementioned metal oxide. Although type of the salt is not particularly limited, chloride salts, bromide salts, and sulfates are preferred. These salts can be used in the form of powder or dispersion.

Although the method for preparing the superparamagnetic particles used in the liposome of the present invention is not particularly limited, they can be prepared, for example, according the method disclosed in Japanese Patent Unexamined Publication (KOHYO, International Patent Publication in Japanese) No. 2002-517085. For example, an aqueous solution containing an iron(II) compound, or an iron(II) compound and a metal(II) compound can be placed under an oxidation condition required for the formation of a magnetic oxide while a pH of the solution is maintained within the range of 7 or higher to prepare iron oxide or ferrite superparamagnetic particles. The superparamagnetic particles used in the liposome of the present invention can also be obtained by mixing an aqueous solution containing a metal(II) compound and an aqueous solution containing iron(III) under an alkaline condition. Furthermore, the method described in Biocatalysis, Vol. 5, pp. 61-69, 1991 can also be used.

In order to form magnetite, for example, it is preferred that iron exists in a solution in two kinds of different oxidation states, namely, as Fe²⁺ and Fe³⁺. Examples of the method for allowing the two kinds of different oxidation states of iron to exist in a solution include a method of adding a mixture of an iron(II) salt and an iron(III) salt, preferably in such a manner that the Fe(II) salt is added in a slightly larger molar amount relative to Fe(III) salt, as compared with a desired magnetic oxide composition, and a method of adding an iron(II) salt or an iron(III) salt and, if necessary, converting a part of Fe²⁺ or Fe³⁺ to the other oxidation state, preferably by oxidation or optionally by reduction.

The resulting magnetic metal oxide is preferably ripened at a temperature not lower than 30° C. and not higher than 100° C., more preferably not lower than 50° C. and not higher than 90° C.

In order to cause interactions between various kinds of metal ions for formation of a magnetic metal oxide, pH of the solution needs to be 7 or higher. The pH can be maintained within the desired range by using an appropriate buffer solution, which is used as an aqueous solution for initial addition of a metal salt, or by adding a base to the solution after the required oxidation state is attained. In order to obtain substantially uniform particle size distribution of the final product, it is preferred that the selected pH value not lower than 7 should be maintained over the whole manufacturing process of the superparamagnetic particles.

The method for preparing superparamagnetic particles may comprise the step of further adding an additional metal salt to the solution for the purpose of controlling the particle size of the superparamagnetic particles. The addition of an additional metal salt to the solution can be performed by, for example, either of the following two kinds of different operation modes. One operation mode is a mode of continuously adding the components (metal salt, oxidation agent and base) to the solution several times, preferably in the same amounts for every addition in a predetermined order, and repeating the foregoing step for a required number of times until a desired particle size of superparamagnetic particles is obtained, so that the particle size is increased stepwise (referred to as the “operation of stepwise mode”). In this mode, the addition amounts of the components for each addition are preferably such amounts that polymerization of the metal ions in the solution (i.e., except for the surfaces of particles) should be substantially avoided.

The other mode is a continuous operation mode, which is a mode of continuously adding the components (metal salt, oxidation agent and base) to the solution at a substantially constant flow rate for each component in a predetermined order to avoid polymerization of the metal ions at a site other than the surfaces of particles.

By using the aforementioned stepwise or continuous operation mode, particles can be formed so as to have a small distribution of the particle size.

When an effective ingredient for MRI such as superparamagnetic particles is contained in the liposome of the present invention, the content thereof is about not less than 10 mass % and not more than 90 mass %, preferably not less than 10 mass % and not more than 80 mass %, more preferably not less than 20 mass % and not more than 80 mass %, based on the total mass of the membrane components.

By using the liposome of the present invention is used, an effective ingredient for MRI such as superparamagnetic particles can be selectively taken up into vascular smooth muscle cells proliferating under influence of foam macrophages. As a result, by using the liposome of the present invention, an MRI imaging with high contrast between a pathological lesion and vascular smooth muscle cells of a non-pathological site is achievable. Therefore, the liposome of the present invention can be suitably used as an MRI contrast medium, in particular, for the imaging of a vascular disease, and the liposome enables, for example, the imaging of arteriosclerotic lesion and restenosis after PTCA.

Although it is not intended to be bound by any specific theory, it is known that, in vascular diseases such as arteriosclerosis or restenosis after PTCA, vascular smooth muscle cells constituting tunica media of blood vessel abnormally proliferate and migrate into endosporium at the same time to narrow blood flow passages. Although triggers that initiate the abnormal proliferation of normal vascular smooth muscle cells have not yet been clearly elucidated, it is known that migration of macrophages into endosporium and foaming are important factors. It is reported that vascular smooth muscle cells then cause phenotype conversion (from constricted to composite type).

Further, as described in J. Biol. Chem., 265, 5226 (1990), for example, it is known that liposomes containing phospholipids, particularly liposomes formed by using PC and PS, likely to accumulate on macrophages with the aid of scavenger receptors. Therefore, it is considered that, by using the liposomes of the present invention, an effective ingredient for MRI such as superparamagnetic particles can be accumulated in a tissue or a lesion in which macrophages localize.

Examples of tissues in which localization of macrophages is observed, which can be suitably imaged by the method of the present invention, include blood vessel, liver, spleen, air vesicle, lymph node, lymph vessel, and renal epithelium. Further, it is known that macrophages accumulate in lesions in certain classes of diseases. Examples of such diseases include tumor, arteriosclerosis, inflammation, infection and the like. Therefore, lesions of such diseases can be identified by using the liposomes of the present invention. In particular, it is known that foam macrophages, which take up a large amount of denatured LDL with the aid of scavenger receptors, accumulate in atherosclerosis lesions at an early stage (Am. J. Pathol., 103, 181 (1981); Annu. Rev. Biochem., 52, 223 (1983)). Therefore, by performing MRI after accumulation of the liposomes of the present invention in the macrophages, it is possible to identify locations of atherosclerosis lesions at an early stage, which is hardly achievable by other means.

The MRI contrast medium containing the liposome of the present invention can be preferably administered parenterally, more preferably administered intravenously. For example, preparations in the form of an injection or a drip infusion can be provided as powdery compositions in a lyophilized form, and they can be used by being dissolved or resuspended just before use in water or an appropriate solvent (e.g., physiological saline, glucose infusion, buffering solution and the like).

A dose of the MRI contrast medium containing the liposome of the present invention can be suitably determined according to properties of effective ingredient for MRI, administration route, clinical indexes, and the like.

### EXAMPLES

The present invention will be explained more specifically with reference to the examples. However, the scope of the present invention is not limited to the following example.

### Example 1

### Preparation of Superparamagnetic Particle Dispersion

Iron(III) chloride hexahydrate (10.8 g) and Iron(II) chloride tetrahydrate (6.4 g) were dissolved in 80 ml of 1 N aqueous hydrochloric acid and mixed. This solution was added with aqueous ammonia (28% by weight, 96 ml) at a rate of 2 ml/minute with stirring. Then, the mixture was heated at 80° C. for 30 minutes, and further cooled to room temperature. The resulting aggregates were washed with water by decantation. Generation of magnetite (Fe₃O₄) having a crystallite size of about 12 nm was confirmed by X-ray diffractometory.

### Example 2

### Uptake Amount of Superparamagnetic Particles by Vascular Smooth Muscle Cells

Dipalmitoyl-PC (Funakoshi, No. 1201-41-0225, 0.73 g) and dipalmitoyl-PS (Funakoshi, No. 1201-42-0237, 0.75 g) were dissolved in chloroform in an eggplant-shaped flask according to the method described in J. Med. Chem., 25 (12), 1500 (1982) to form a uniform solution. Then, the solvent was evaporated under reduced pressure to form a thin membrane on the bottom of the flask. The dispersion prepared in Example 1 was heated at 65° C., and the heated dispersion (10 ml, 10 mM) was mixed with the aforementioned thin membrane. This mixture was heated at 65° C. for 15 minutes with stirring, and subjected to ultrasonication (No. 3542 probe type oscillator, Branson, 0.1 mW) for 5 minutes under ice cooling to obtain a uniform liposome dispersion. The diameters of the liposomes in the resulting dispersion were measured by using WBC analyzer (A-1042, Nihon Kohden). As a result, the diameters were found to be 40 to 65 nm. The liposome preparation prepared by the above method was added to a mixed culture system of vascular smooth muscle cells and macrophage described in International Publication WO 01/82977. The cells were cultured at 37° C. under 5% CO₂ for 24 hours. As a result, uptake of the superparamagnetic particles into the vascular smooth muscle cells could be confirmed. As described above, the liposome of the present invention was efficiently taken up by vascular smooth muscle cells, and it is clearly understood that the liposome has superior properties as an MRI contrast medium.

### Comparative Example 1

The amounts of phospholipids used in Example 2, dipalmitoyl-PC and dipalmitoyl-PS (Funakoshi, No. 1201-42-0237), were changed to 0.73 g and 0.075 g, respectively. This means PC:PS=10:1 in a molar ratio. Except for the above, uptake amount into vascular smooth muscle cells was measured under the same conditions as those of Example 2. In this experiment, only a trace amount of the superparamagnetic particles were taken up into vascular smooth muscle cells.

### Comparative Example 2

Uptake amount into vascular smooth muscle cells was measured under the same conditions as those of Example 2 except that the superparamagnetic particles were changed to superparamagnetic particles having a particle size of 100 nm. In this experiment, only a trace amount of the superparamagnetic particles were taken up into vascular smooth muscle cells.

1. A liposome containing a phosphatidylcholine and a phosphatidylserine in combination as membrane components at a phosphatidylcholine:phosphatidylserine molar ratio of 3:1 to 1:2.

2. The liposome according to claim 1, wherein the molar ratio of phosphatidylcholine and phosphatidylserine is 1:1.

3. The liposome according to claim 1, which contains superparamagnetic particles having a mean particle size not less than 1 nm and not more than 50 nm.

4. The liposome according to claim 3, wherein the superparamagnetic particles are selected from the group consisting of particles of iron oxide and ferrite (Fe, M)304.

5. The liposome according to claim 3, wherein the superparamagnetic particles are particles of magnetite, maghemite, or a mixture thereof.

6. An MRI contrast medium comprising the liposome according to claim 1.

7. The MRI contrast medium according to claim 6, which is used for imaging of a vascular disease.

8. The MRI contrast medium according to claim 6, which is used for imaging of vascular smooth muscle cells which abnormally proliferate under an influence of foam macrophages.

9. The MRI contrast medium according to claim 6, which is used for imaging of a tissue or lesion where macrophages localize.

10. The MRI contrast medium according to claim 9, wherein the tissue where macrophages localize is selected from the group consisting of tissues of liver, spleen, air vesicle, lymph node, lymph vessel, and renal epithelium.

11. The MRI contrast medium according to claim 9, wherein the pathological site where macrophages localize is selected from the group consisting of lesions of tumor, inflammation, and infection.

12. The liposome according to claim 2, which contains superparamagnetic particles having a mean particle size not less than 1 nm and not more than 50 nm.

13. An MRI contrast medium comprising the liposome according to claim 2.

14. An MRI contrast medium comprising the liposome according to claim 3.

15. An MRI contrast medium comprising the liposome according to claim 4.

16. An MRI contrast medium comprising the liposome according to claim 5.

17. An MRI contrast medium comprising the liposome according to claim 12.
